(19) United States
(12) Patent Application Publication
(10) Pub. No.: US 2004/0062789 A1
(43) Pub. Date: Apr. 1, 2004

(54) Compacted biomaterials

(75) Inventors: Ian MacMillan Ward, Leeds (GB); Noe Hugo Ladizesky, Salta (AR)

Correspondence Address: NIXON & VANDERHYE, PC, 1100 N GLEBE ROAD, 8TH FLOOR, ARLINGTON, VA, 22201-4714, US

(73) Assignee: BTG International Limited, London (GB)

(21) Appl. No.: 10/673,259

(22) Filed: Sep. 30, 2003

Related U.S. Application Data
(63) Continuation of application No. 09/447,738, filed on Nov. 23, 1999, now abandoned.
Application No. 09/447,738 is a division of application No. 09/137,127, filed on Aug. 20, 1998.

(30) Foreign Application Priority Data
Jan. 15, 1996 (GB) 9600800.8
May 30, 1996 (GB) 9611249.5
Jan. 14, 1997 (WO) PCT/GB97/00105

(51) Int. Cl. A61K 33/42

(52) U.S. Cl. 424/423; 424/602

(57) ABSTRACT

A composite material comprising an inorganic filler material and a oriented fibrous polymeric material characterised in that the fibrous material has areas of adjacent fibres fused together to form a network or continuous matrix while retaining oriented fibrous structure in the composite.

The present invention relates to novel composite materials, to a process for production of such materials and to their use as structural materials, particularly in prostheses but also in other non-physiological situations.

Hydroxyapatite (HA) reinforced high density polyethylene (PE) composite (HAPEX) was pioneered as a bone substitute by Bonfield et al. (see GB 2085461 A) who demonstrated that an optimum combination of mechanical and biological performance is produced by HA content in the region of 40 vol %. This amount of reinforcement was shown to result in a bone substitute with a stiffness and strength suitable for minor load bearing applications. However, for major load bearing skeletal implants considerably higher stiffness and strength are required, comparable with the values associated with cortical bone.

GB 2085461A describes a composite material comprising a homo or co-polyolefin and up to 80% by volume of a particulate inorganic solid for use as an endoprosthesis.

GB 1452654 relates to a moulded bonded non-woven fibrous product comprising an open web of two heat stabilised crimped fibres of different softening point, which have been compressed at a temperature in excess of the softening temperature. That document teaches that active particles of active or conductive carbon or ion exchange resins may be incorporated into the product and describes its use in toys, pillows, mattresses, upholstery, filtration media and air flow stabilisers.

EP 0116845A discloses a method for consolidating polyethylene fibre networks comprising compacting them at 100° C. to 160° C. at a pressure sufficient to cause them to adhere. Mineral filled fibres are stated to be amenable to this treatment.

U.S. Pat. No. 5,133,835 discloses a heat bonded non-woven composite web, eg. in sheet form, comprising three wet laid fibre types, each of independent melting point, which optionally contains inorganic filler and describes its use as high strength printable protective wrapping.

U.S. Pat. No. 4,516,276 discloses a bone substitute material comprising a lyophilized collagen fleece and powdered or granular apatite. Fusion of collagen fibres is not taught.

WO 9411556 discloses a non-woven web comprising immobilized particulate matter which does not extend into its upper and lower surfaces for use in chemical defence.

It is desirable to have methods for the production of suitable structural materials, and particularly bio-structural materials, with the purpose of achieving increased bio-compatible filler content, particularly HA content for higher bioactivity, and better mechanical properties such as non-brittleness while having a load bearing capability.

The present inventors have now provided such a method having the basic concept of combining polymeric fibres, preferably of polyolefins such as PE, with fillers, particularly biocompatible fillers such as HA, to produce structural materials, particularly bone analogues, by replacing the previously used isotropic polymer with polymeric fibre, particularly high modulus polyolefin fibre such as polyethylene (HMPE) fibres, and compressing this mixture using hot compaction. Preferably the fibre is used as pieces of fibre in chopped fibre form.

Molecular orientation in a polymer leads to a significant enhancement in the stiffness and strength along the orientation direction (I. M. Ward, “The preparation, structure and properties of ultra-high modulus flexible polymers”, Advances in Polymer Science, vol. 70, pp. 1-70, 1985).

Hot compaction is a process which allows the production of large section polymeric products with substantial fibre morphology content, retaining to a large extent the high stiffness and strength associated with fibres (see GB 2253420, U.S. Ser. No. 07/934,500 and No. 08/315,680 all derived from WO 92/15440), particularly high modulus fibres. The process is defined in WO 92/15440 as a process in which an assembly of oriented polymer fibres is maintained in intimate contact at an elevated temperature sufficient to melt a proportion of the polymer and subsequently compressed. The elevated temperature is taught to preferably be at least that at which an extrapolation of the leading edge of the endotherm of the oriented fibres measured by differential scanning calorimetry intersects the temperature axis, and more preferably is less than the peak temperature of melting of the polymer fibres as so measured.

This process, by control of heat and pressure, allows preferential surface melting of fibres. Thus the polymer melted includes that at the surface and preferably is between 5% and 20% by weight of the fibre and preferably no more than 10%, although up to 50% melting may be useful. The amount melted should ideally be enough to fill the spaces between fibres upon compaction such that trapped air may be avoided, but not so much that fibre morphology, particularly orientation, is lost. Fibre diameters are said to be typically between 0.005 and 0.05 μm

Other literature relating to this process includes “The hot compaction of high modulus melt-spun polyethylene fibres”, Hine et al., Jnl. Materials Science 28 (1993), 316-324; “Morphology of compacted polyethylene fibres”, R. H. Olley et al., Jnl. Materials Science 28 (1993), 1107-1112; “Compaction of high-modulus melt-spun polyethylene fibres at temperatures above and below optimum”, M. A. Kabeel et al., Jnl. Materials Science 29 (1994), 4694-4699; “Differential melting in compacted high-modulus melt-spun polyethylene fibres”, M. A. Kabeel et al., Jnl. Materials Science 30 (1995), 601-606; “The hot compaction of polyethylene terephthalate”, J. Rasburn et al., Jnl. Materials Science 30 (1995), 615-622; “The hot compaction of polypropylene fibres”, M. I. Abo El-Matty et al., Jnl. Materials Science 31 (1996), 1157-1163.

The present method is of particular interest in the fabrication of HA/PE composites having a fibre morphology matrix using chopped HMPE fibres. The present inventors have further found, surprisingly, that such a material is amenable advantageously to extrusion, particularly hydrostatic extrusion, a technology never before attempted with hot compacted fibres. While the present method may be preferentially applied to HMPE polymers, it may also be applied to other oriented polymers such as vinyls, polyesters, polyamides, polyetherketones and polyacetals such as vinyl chlorides, vinyl fluorides, vinylidene fluorides, PHB, PEEK, polyoxymethylenes and all the other materials referred to as suitable for hot compaction use in WO 92/15440 and the above prior art.

Thus in a first aspect of the present invention there is provided a composite material comprising an inorganic filler material and a fibrous polymeric material characterised in that the fibrous material comprises oriented polymeric fibres and has areas of adjacent oriented fibres fused together to form a network or continuous matrix while retaining the fibrous structure in the composite. This structure will of course comprise oriented fibre.

Preferably the inorganic filler is a particulate filler. Examples of fillers include silicas, talc, mica, graphite, metal oxides, metal hydroxides and metal carbonates. Most preferably the inorganic filler is a biocompatible material, such as for example an apatite, eg. hydroxyapatite, a biocompatible calcium phosphate ceramic. The amount of filler is preferably up to 60% vol. of the material, more preferably from 20 to 50% vol.

Increasing hydroxyapatite content leads to reduced die swell effect when the material is of extruded form. The composite material is preferably of extruded form and particularly of a hydrostatically extruded form. It is found that extrusion induces orientation of the material, increases its melting point and improves mechanical properties. Increased extrusion ratio lowers die swell.

Preferred composite materials of the invention have a flexural modulus between 7 and 30 GPa, preferably greater than 10 GPa, still more preferably having a flexural modulus greater than 12 GPa, most preferably greater than 15 GPa

Preferred composite materials of the invention have a flexural strength between 50 and 150 Mpa, more preferably greater than 60 Mpa, still more preferably greater than 80 Mpa and most preferably greater than 100 MPa.

Preferred composite materials of the invention have a flexural ductility between 0.5 and 10%, more preferably, between 0.5 and 7%, most preferably between 0.5 and 4%.

Preferably the fibrous polymeric material is a polyolefin, preferably polypropylene or polyethylene, most preferably polyethylene, and most preferably is in a high modulus form. The polymer may be in the form of discrete fibres or as a fabric or web, which may be woven or non-woven. Preferably the material includes a recrystallized melt phase of melting point less than that of the starting material fibres which binds them together.

The fibre or web or fabric may be in divided form, eg. chopped or otherwise cut into sections, eg. of 0.1 to 10 mm in length, more preferably 0.5 to 5 mm in length. The most preferred forms of the combined fibre and filler have been powderized and recompacted thus giving a more homogeneous structure. Powderizing to a maximum dimension of 0.1 to 1 mm diameter is convenient, with 0.5 mm being typical.

In a second aspect of the invention there is provided a method for producing a composite material, eg. a structural material, comprising combining oriented polymeric fibres with an inorganic filler material comprising compressing the combined material using hot compaction.

Particularly the method produces a composite material from an inorganic filler material and a fibrous polymeric material which comprises oriented polymeric fibres by steps of mixing and heating the filler material and fibrous polymeric material and is characterised in that it comprises (i) combining the materials and maintaining them at a contact pressure such that at least some of the fibres are in intimate contact with each other, (ii) heating the combined materials so maintained at a temperature and for a time such as to melt a proportion of the fibre and (iii) compressing the heated mixture at a compaction pressure.

The combining of the materials is preferably achieved by mixing while the proportion of the fibre that melts is less than the whole such that oriented fibre morphology is maintained in the product. As described in the prior art, the proportion of the fibre that melts in hot compaction will include that at surface and preferably the part of the fibre surface that melts is from 5 to 95% of the fibre, more preferably from 5 to 50% of the fibre and most preferably 5 to 10%. Preferably the fibres are fused in such a manner that there are substantially no voids in the material.

Preferably the material is cooled after compaction such that on cooling the melted part of the fibrous polymeric material forms a three dimensional matrix binding the fibrous material and filler material together. In the contact pressure maintaining step the mixture is preferably maintained at a temperature at least that which an extrapolation of the leading edge of the endotherm of the fibrous material measured by differential scanning calorimetry intersects the temperature axis.

Preferably the temperature at which the combined materials are maintained is less than the peak temperature of melting of the polymer fibres as measured by differential scanning calorimetry.

As claimed in U.S. Ser. No. 08/315,680, the temperature is preferably sufficient to selectively melt polymer which on cooling recrystallizes to form a melt phase which has a melting point less than the melting point of the starting material fibres and which binds these fibres together in the product.

The combined material, eg mixture, is preferably maintained at a contact pressure of 0.5 to 4 MPa during step (i) and step (ii) prior to compressing at a compaction pressure; still more preferably between 0.5 and 2 MPa prior to compressing at a compaction pressure. There may be a single compaction step, particularly at the contact pressure of step (i) and (ii) only where this is a compaction pressure sufficiently low to allow preferential surface melting of the fibres.

The temperature at which the combined material, eg. mixture, is maintained is preferably at between 1 and 10° C. below the melting point of the polymeric material, more preferably between 1 and 5° C. below the melting point of the polymeric material.

In a particularly preferred method of the invention the compacted material produced in step (iii) is subjected to extrusion, more preferably hydrostatic extrusion. In a preferred such process the product from step (iii) or from the extrusion step is advantageously powderized then reprocessed as in steps (i) to (iii). This reprocessing may be carried out more than once and is preferably carried out by recompacting at a temperature of a few degrees centigrade lower than the first compaction in order to ensure that only the originally melted fraction is re-melted and the fibre morphology is maintained. A typical recompaction temperature is about 4° C. less than the first melting temperature.

Preferably the reprocessed material is then subjected to extrusion, preferably hydrostatic extrusion.

Extrusion may be carried out using any suitable pressurising method to force the material through a die. Hydrostatic extrusion is preferably performed by (iv) placing a billet of the material in contact with a die orifice while being surrounded by a fluid medium, (v) heating then fluid and the billet to a temperature below the melting point of the polymeric component of the material and (vi) applying pressure to the fluid such as to cause the billet to be extruded through the die.

Preferably the die is a convergent die, more preferably having an extrusion ratio of extruded product 3:1 or more, more preferably 7:1 or more and most preferably at least 11:1.

Preferably the fluid used in the hydrostatic extrusion is an oil. The extrusion heating step may be effected at the compaction temperature thus allowing one step compaction and extrusion or even continuous contact, compaction and extrusion.

For all these processes the compaction pressure used in step (iii) is preferably from 5 to 1000 MPa, more preferably 20 to 500 Mpa, and most preferably from 40 to 80 MPa.

Preferably the polymer is a homo or co-polymer of a polyolefin, more preferably having a weight average molecular weight of 50,000 to 3,000,000 and still more preferably from 100,000 to 3,000,000 and most preferably 500,000 to 3,000,000. Polymers of 10,000 to 400,000 molecular weight, e.g. 50,000 to 200,000 may also conveniently be used.

The fibre is preferably gel or melt spun fibre.

The composites of the invention are preferably used in or as prostheses, and particularly as bone replacement prostheses.

The method and materials of the present invention will now be described by way of illustration only by reference to the following non-limiting Examples. Further embodiments falling within the scope of the invention will occur to those skilled in the art in the light of these.

### EXAMPLES

Experimental

Materials

HA, a synthetic calcium phosphate ceramic [Ca₁₀(PO₄)₆(OH)], was supplied by Biotal Ltd., UK at Grade P88 having a 4.14 μm average size. Chopped HMPE fibres were supplied by Hoechst Celanese Research Co. (Summit, N.J., USA), produced from continuous fibres manufactured by SNIA Fibres (Cesano Maderno, Italy). Some samples also contained a third type of material, namely a 40 vol % HA/60 vol % PE composite produced with melt compounding technology. The polymer used in this material was Rigidex HM 4560 PX (BP Chemicals Ltd., UK) whilst the HA was P88 grade.

Table 1 gives the main parameter values characterising the PEs and HA used in this work.

Methodology

Preparation of Composites

The HA particles and chopped fibres were mixed at room temperature with a Braun Hand Blender MR 350 with the chopper HC accessory (Braun (UK) Ltd., London). This equipment was found particularly convenient because the hand held motor casing and the chopper are both axially aligned, allowing the blending to be carried out at an inclined angle while rolling the lower end on the bench. This procedure assisted the movement of the fibres inside the containers and avoided agglomeration. The chopper accessory was modified to improve efficiency as follows: a) a second pair of blades was added midway along the shaft, b) the nylon bearings were replaced by ball bearings and c) the plastic base was replaced by a heavier aluminium base. Mixing of about 5 g of chopped fibres plus the required amount of HA was carried out for about 3 minutes using the three available motor speeds and rest periods in a reproducible sequence.

The composites were compacted in an aluminium mould placed in a hydraulic hot press. The mould produced samples of rectangular cross section and 150×10 mm L×W. Maximum thickness was 8 mm. The temperature during compaction was monitored with a probe connected to an electronic thermometer. 5 mm of the sensing end of the probe could be inserted with a tight fit into holes bored at various points around the mould. The space between the hot plates of the press was shielded from the outside with Perspex sheets. With these arrangements a predetermined moulding temperature (usually around 135 C) in the middle of the mould could be achieved within 0.2° C., whilst the gradient between the two ends of the mould was 0.5° C.

The blended material inside the mould was maintained at the predetermined temperature for 20 minutes under a small pressure (approximately 100 psi=0.69 MPa) to ensure good thermal contact between the mould and the two hot plates of the press. The compression load was then increased rapidly to 9 tonne giving a compaction pressure of about 60 MPa, followed by switching the heating off and water cooling of the hot press. The mould was allowed to cool and reached a temperature of 50° C. in about 30 minutes, whereafter it was left on a bench to cool down to room temperature before removing the sample.

A small number of samples with 30 vol % HA (as supplied) were prepared using hydroxyapatite particles and HMPE chopped fibres mixed with an alternative technique. This is referred to as “liquid nitrogen” blending. Clusters of chopped fibres as supplied were first opened up in a Waring 8011 G Rotary Blender (Waring Products Div., Dynamics Corporation of America, Connecticut 06057, USA) fitted with the stainless steel mini container type MC3. Once this stage had been completed, the required amount of hydroxapatite was placed in a stainless steel saucepan with about 300 cc liquid nitrogen. The mixture became whitish and homogeneous but after about 30 seconds the ceramic particles precipitated to the bottom of the saucepan. The chopped fibres were incorporated into the liquid whilst in the whitish stage, ensuring a continuous and gently boiling action; if necessary, the saucepan was placed on a warm plate. When all the liquid nitrogen had been evaporated, the hydroxyapatite particles remained fully incorporated into the network of fibres. Room temperature blending involved some wastage of hydroxyapatite because particles escaped as dust and, in addition, a small amount of powder was left unmixed in the container. This wastage was qualitatively taken into account by starting the mixing with a small excess of the hydroxyapatite.

The composites were compacted in an aluminium mould placed in a hydraulic hot press. The samples were 150×10 mm rectangular bars, whilst the thickness varied between 2 and 8 mm, according to the sample use. The temperature during compaction was monitored with a probe connected to an electronic thermometer, inserted as a tight fit into holes bored at various points on the mould. The space between the hot plates of the press was shielded with Perspex sheets. Thus a predetermined moulding temperature (usually around 136.0° C.) in the middle of the mould could be achieved within 0.3° C., whilst the gradient between the two ends of the mould was 0.5° C.

The blended material inside the mould was maintained at the predetermined temperature for 20 minutes under low pressure to ensure good thermal contact between the mould and the two hot plates of the press. The compression load was then increased rapidly to 9 tonne (60 MPa pressure), when the heating was switched off and water cooling of the hot press started. The mould was maintained at constant pressure until it reached a temperature of 50° C., about 30 minutes, after which it was left on a bench to cool to room temperature before removing the sample.

Some samples were powderised to improve the HA distribution within the polymeric matrix. The powderising process included three stages a) crushing in a fly press, b) chopping with a Kenwood Chef Food Mixer fitted with the spice mill attachment (Kenwood Ltd., Havant, Hampshire, UK), and c) powderising proper in a Fritsch Pulverisette 14 Rotor-Speed Mill (Fritsch GmbH, Idai-Oberstein, Germany) using progressively finer sieves from 6 mm down to, when required, 80 μm. The Rotor-Speed Mill was fitted with a 12 knives stainless rotor and the speed used was 16,000 r.p.m.

Powderising could be readily achieved down to a 1 mm sieve. Use of finer sieves required considerable care owing to heat generation. The material to be fed into the Rotor-Speed mill was kept in a beaker, which was itself immersed in liquid nitrogen. No liquid nitrogen was poured into the machine.

After powderising, the material was re-compacted. At this stage it was necessary to re-melt the polyethylene fraction which melted during the first compaction, but without affecting the fraction with fibre morphology. This was achieved by re-compacting at 3° C. to 4° C. lower than the temperature used during the first compaction as the HMPE fibres melt at about 140° C., which compares with ≈130° C. for isotropic PE.

In some cases, composites were prepared in a still further different way, namely the chopped HMPE fibres were blended with a mixture of HA and compacted, 40 vol % HA/60 vol % PE composite. The latter was prepared as seen in GB 2085461 B and was available in coarse powder form. The HA and compounded composite were blended in a Waring 8011G Rotary Blender (Waring Products Div., Dynamics Corporation of America, Connecticut, 06057, USA) fitted with the stainless steel mini container MC3. The mixing was carried out using a reproducible sequence of blending pulses (15 seconds), tapping and scrubbing the containers floor with a metal spatula. The powder thus obtained was compressed in a stainless steel mould placed in a hydraulic press. The procedure for this stage was similar to that followed for the production of compacted fibre composites, as seen above, except that the moulding temperature was 180° C. and the samples were cylindrical rods 60 mm×12 mm or 60 mm×18 mm, L×diameter. For convenience this material will be referred to as “enriched” compounded composite (ECC).

The rods of “enriched” compounded composite were powderised as seen above and blended with the chopped HMPE fibres following a similar technique as used for the HA/chopped fibre system. The proportion of the various materials used was chosen such that the final mix achieved the predetermined HA content, while two thirds of the PE had fibre morphology.

Hydrostatic Extrusion

Details of the experimental procedure may be seen in Gibson and Ward (see Example 2) and only a brief summary will be presented here.

The die used had a cone semi-angle of 15° and the bore diameters were 1.8 mm, 2.5 mm or 3.5 mm, according to the extrusion ratio (ER) and the original dimensions of the compacted bars of composite to be extruded. Billets were machined from the bars with a 15° nose to create an initial pressure seal. At the end of the nose a constant diameter stub was also machined, which protruded a few millimetres through the die. A cable attached to the stub was used to drive a rotary potentiometer to provide a displacement signal which was recorded from the beginning of the extrusion (this was the first time that the HE process was monitored from its initial stages). A haul off load of 100 g was attached to the free end of the cable to ensure a firm drive of the rotary potentiometer. The back 3 mm of the billet was machined to a larger diameter to act as a plug and prevent the violent release of pressure at the end of a run.

The pressurising fluid was castor oil (J. L. Seaton, Hull, UK). The billets were coated with two layers of Evostick (Evode Ltd., UK) to avoid direct contact between the polymer matrix and the pressurising fluid, which involves a risk of stress cracking. It was found that the Evostick coating peeled off during extrusion and did not go through the die.

After preliminary trials, the extrusion temperature was fixed at 115° C. The extrusion pressure was a function of the material and the extrusion ratio (ER, ratio of the initial and final cross sections). There was little control of the extrusion rate, which was about 1.5 mm min⁻¹.

Differential Scanning Calorimetry (DSC)

The effect of the various processing stages (blending, compaction, powderising, re-compaction and HE) on the morphology of the PE matrix was qualitatively assessed by studying the melting behaviours of the material. For this purpose, a Perkin Elmer: Differential Scanning Colorimeter DSC7 (Perkin Elmer Corp., Norwalk, Conn., USA) was used with a scanning rate of 10° C. and 2 to 10 mg of material for each run.

Scanning Electron Microscopy (SEM)

The effect of the various processing stages on the dispersion of HA in the PE matrix was assessed with SEM techniques. The specimen preparation procedures consisted of sectioning, moulding in an acrylic resin, grinding on silicon carbide papers from grade 220 down to 1000 grit, polishing using alumina powders with a particle size of 5, 1 and 0.3 μm progressively, cleaning in an ultrasonic bath to remove the alumina powder from the polished surface, drying with compressed air and gold coating with a thickness of approximately 20 rm. The polished surfaces were examined under a JEOL 6300 SEM

Mechanical Testing:

The mechanical properties of the composites were assessed in flexural (F, three point bending) mode of deformation. Three main constants were measured; modulus (FM), strength (FS) and ductility (FD). All the mechanical tests were carried out at room temperature (22+1.50° C.) using an Instron machine (Instron Ltd., High Wycombe, UK).

Flexural Measurements

Rectangular bars and cylindrical rods were tested in three point bending using identical parameters. These are shown in Table 3. Broadly, plates were tested following ASTM 790 recommendations, while rods were tested with their original extruded diameters.

The formulae used to calculate the various flexural properties are those obtained from the simple beam theory. For convenience, these are shown in Table 4. Note that the terms “stain” and “ductility” refer to the maximum strain (in %) in the sample at a given deflection. For all the samples the following conditions applies:

$$\frac{Gauge length}{Thickness} \geq 10$$

as required by the simple beam theory in order to neglect shear deformation. Preferably this is above 15.

Some rods did not break in bending but, instead, the load deflection curve exhibited a yield point. Unless otherwise stated, FD was measured as the maximum strain at the deflection, after the yield point, giving 25% decrease from maximum load.

All materials produced were evaluated in terms of their flexural stiffness, strength and ductility. A qualitative assessment of the effect of the various processing stages on the matrix morphology was made with differential scanning calorimetry (DSC). The dispersion of the filler, ie. HA in the fibre matrix, ie. chopped PE, was studied with scanning electron microscopy (SEM) technique.

TABLE 1
MATERIAL CHARACTERIZATION

| HA (P88) | |
|---|---|
| Density (g/cm³) | 3.156 |
| Particle size (μm) | 4.14 |
| HMPE Fibre | |
| Density (g/cm³) | 0.960 |
| \overline{M}_{W} | 130,000 |
| \overline{M}_{N} | 12,000 |
| DR | 30:1 |
| Diameters (μm) | 13 |
| Tensile modulus (GPa) | 40 |
| Tensile strength (GPa) | 1.3 |
| Fracture strain | 5% |
| Segment length of chopped fibre (mm) | 3.2-3.8 (3.5) |
| Rigidex HM 4560XP | |
| Density (g/cm³⁾ | 0.945 |
| \overline{M}_{W} | 225,000 |
| \overline{M}_{N} | 24,000 |
| Tensile modulus (GPa) | 0.68 |
| Tensile strength (MPa) | 23.5 |
| Fracture strain % | >300 |

TABLE 2
FLEXURAL TEST PARAMETERS

| | LOADING/ | | NOMINAL SAMPLE DIMENSIONS | | | | |
|---|---|---|---|---|---|---|---|
| | SUPPORTING | | (mm) | | | | PROPERTY |
| | RODS | GAUGE | | CROSS SECTION | | CROSSHEAD | CALCULATED |
| | DIAMETER | LENGTH+ | | RECTANGULAR | CYLINDRICAL | SPEED | AT STRAIN |
| TEST | (mm) | (mm) | LENGTH | BARS | RODS | (mm/min) | (%) |
| FM₁₃₀ | 25/12 | 130 | 150 | 10 × 2 | As Extruded | 2.0 | 0.03 |
| FM₅₀ | 10/10 | 50 | 75 | | | 0.5 | 0.03 |
| FS/FD | 10/10 | 50 | 75 | | | 2.0 | — |

+Distance between the supporting rods

TABLE 3
SIMPLE BEAM THEORY FORMULAE FOR
THREE POINT BENDING

| | RECTANGULAR BARS | CYLINDRICAL RODS |
|---|---|---|
| FM | $\frac{1}{4} \frac{{WL}^{3}}{{δbd}^{3}}$ | $\frac{4}{3} \frac{{WL}^{3}}{πδD}$ |
| FS | $\frac{3}{2} \frac{WL}{{bd}^{2}}$ | $\frac{8 WL}{{πd}^{3}}$ |
| MAXIMUM STRAIN | $6 \frac{d δ}{{L}^{2}}$ | $\frac{6 D δ}{{L}^{2}}$ |
| SYMBOLS | W = Load | W = Load |
| | δ = Deflection | δ = Deflection |
| | L = Gauge Length | L = Gauge Length |
| | d = Sample Thickness | d = |
| | | Diameter of Overall Sample |
| | b = Sample Width | D = |
| | | Diameter of Gauge Length |

### Example 1

### Hydroxyapatite/High Modulus Polyethylene Fibre Composite

HA particles and chopped HMPE fibres were mixed at room temperature with a modified domestic hand blender. The composites were compacted in an aluminium mould placed in a hydraulic hot press with preliminary experiments showing that compaction temperatures of between 137.0° C. and 138.0° C. are adequate to melt a small proportion of the fibre surface, as described in GB 2253420, and form a continuous network of recrystallized PE binding the fibres and the HA particles together. This recrystallized PE may contain voids.

Some samples were powderised after compaction with the purpose of improving the HA distribution within the polymeric matrix and reducing or eliminating voids. The material was recompacted at about 3.0° C.-4.0° C. lower than the first compaction temperature, ensuring the re-melting of the PE fraction melted during the first compaction, but with a minimum effect on the fraction retaining the fibre morphology, the HMPE fibres melt at ≅140° C., which compares with ≅130° C. for the re-crystallized PE, ie. that which did not previously melt.

In some cases the composites were prepared in a somewhat different way, namely by blending the HMPE chopped fibres with a mixture of HA and HAPEX (40 vol % HA) whereby void formation is reduced or eliminated. This mixture is referred to as “enriched” HAPEX. The proportions of the various materials used were chosen such that the final mix had the predetermined HA content, with two-this of the PE having fibre morphology. A compaction temperature of about 0.5° C. lower was used for this material.

Liquid nitrogen blending was also used for some samples as described previously above.

### Example 2

### Hydrostatically Extruded Hot Compacted Composite

The hydrostatic extrusion process [A. G. Gibson and I. M. Ward, “Hydrostatic extrusion of linear polyethylene: effects of molecular weight and product diameter”, J. Polym. Sci., Polym. Phys. Ed., vol. 16, pp. 2015-3030, 1978] used a billet of the composites prepared above surrounded by a fluid which was heated up below its melting point. The billet was made to pass through a convergent die by the application of a back pressure to the fluid. The extrusion ratio (ER) is defined as the ratio of the cross section area of the billet to that of the die bore. The extrusion temperature used was about 115° C. in each case; this being higher than the 100° C. used for linear polyethylene by Gibson and Ward.

It was found that powderising and re-compaction were preferred requirements for the successful hydrostatic extrusion of HA/chopped HMPE fibre composites.

The mechanical properties of the composites were assessed in three point bending. Three measurements were undertaken, namely flexural modulus (FM), flexural strength (FS) and flexural ductility (FD). The term “ductility” refers to the maximum stain (in %) in the sample at the deflection producing failure, i.e. at maximum load.

Properties:

Tables 4a and 4b give the main flexural properties of HA/chopped HMPE fibre composites. For comparison, some results obtained with other materials are also included. All the hydrostatically extruded composites achieve the levels of stiffness and strength associated with cortical bone. None of the composites produced without hydrostatic extrusion possess properties comparable to the biological tissue.

When comparing the system without hydrostatic extrusion, Table 4a shows that the properties of HAPEX are broadly matched by the properties of HA/chopped fibre composites. However, after hydrostatic extrusion these systems are distinctly superior to extruded HAPEX (Table 4b).

SEM observations with polished samples showed that HAPEX has a highly homogeneous distribution of HA particles in the polymeric matrix, whereas HA/chopped fibre composites have regions with varying degrees of HA content and voids. Powderising and re-compaction of the HA/chopped fibre systems significantly improved their HA distribution and reduced or eliminated voids. These stages were usually required for successful hydrostatic extrusion, as noted above.

Table 4a shows that powderising and recompaction of HA/chopped fibre composites are accompanied by a reduction in their stiffness and strength. This can be attributed to damage of the fibre morphology taking place during the powderising stage, as shown by DSC studies, which also reveal that a high melting point morphology (fibre morphology) is reestablished during the hydrostatic extrusion process, accounting for the superior properties of these systems (Table 4b).

Thus these results show that hydrostatic extrusion of hot compacted HA/chopped HMPE fibre composites which have been powderised and re-compacted provides the highest stiffness and strength yet encountered with HA/PE bone substitute material, fully comparable with the values associated with cortical bone.

Generally, when compaction temperature was lowered, inhomogeneous powdery samples resulted while high temperatures, eg. 140° C., led to decreased stiffness and strength. Powderising and recompacting of samples gave significant decrease in scatter of results. Extrusion of composites of 50 volume % HA or more could only be achieved after powderising with 80μ sieves.

TABLE 4
Flexural properties of extruded and non-extruded HA/chopped HMPE
fibre composites of the invention and comparative bone and HAPEX.
4(a) Non-extruded (Example 1)

| HA content | Powderising | FM | FS | FD |
|---|---|---|---|---|
| [vol %] | [mm] | [GPa] | [MPa] | [%] |
| Cortical bone | — | 7-30 | 50-150 | 0.5-3.0 |
| HAPEX | — | 4.7 | 32 | 1.4 |
| 0 | — | 3.9 | 54 | 4.2 |
| | 0.5 | 2.4 | 19 | 0.9 |
| 20 | — | 5.8 | 49 | 2.8 |
| | 0.5 | 3.6 | 41 | 2.7 |
| 30* | — | 5.5 | 47 | 2.3 |
| | 0.5 | 4.2 | 38 | 1.9 |
| 40 | 0.08 | 7.4 | 36 | 0.8 |

mm = sieve perforation
HAPEX = 40 vol % HA
*= HA incorporated as enriched HAPEX

TABLE 4(b)
Hydrostatically extruded (Example 2).

| HA content | | FM | FS | FD |
|---|---|---|---|---|
| [vol %] | ER | [GPa] | [MPa] | [%] |
| HAPEX | 8:1 | 8.8 | 81 | 5.5 |
| 0 | 4:1 | 6.9 | 88 | 6.0 |
| | 7:1 | 12.7 | 103 | 3.9 |
| 20 | 7:1 | 15.2 | 119 | 3.6 |
| 30 | 4:1 | 9.3 | 86 | 3.4 |
| | 7:1 | 15.5 | 104 | 3.0 |
| | 11:1 | 19.5 | 117 | 2.8 |
| 30* | 4:1 | 9.0 | 75 | 3.6 |
| | 7:1 | 11.9 | 87 | 3.3 |
| | 11:1 | 15.2 | 113 | 2.8 |
| 40 | 4:1 | 10.8 | 86 | 3.8 |
| | 7:1 | 14.1 | 97 | 3.8 |
| 50* | 7:1 | 12.4 | 72 | 2.8 |
| | 11:1 | 13.5 | 67 | 1.6 |

*For these systems the HA was incorporated as “enriched” HAPEX.

1. A composite material comprising an inorganic filler material and a fibrous polymeric material characterised in that the fibrous material comprises oriented polymeric fibres and has areas of adjacent oriented fibres fused together to form a network or continuous matrix while retaining fibrous structure in the composite.

2. A composite material as claimed in claim 1 wherein the fused fibres are in chopped form.

3. A composite material as claimed in claim 1 or claim 2 being of a substantially void free form.

4. A composite material as claimed in any one of claims 1 to 3 wherein the inorganic filler is a particulate filler.

5. A composite material as claimed in any one of claims 1 to 4 wherein the filler is selected from talc, mica, graphite, metal oxides, metal hydroxides, carbonates and phosphates.

6. A composite material as claimed in any one of claims 1 to 5 wherein the inorganic filler is a biocompatible material.

7. A composite material as claimed in claim 6 wherein the biocompatible material is an apatite.

8. A composite material as claimed in claim 7 wherein the apatite is hydroxyapatite.

9. A composite material as claimed in any one of claims 1 to 8 wherein the material is of extruded form.

10 A composite material as claimed in claim 9 wherein the material is in hydrostatically extruded form.

11. A composite material as claimed in any one of claims 1 to 10 having flexural modulus between 7 and 30 GPa.

12. A composite material as claimed in claim 11 having flexural modulus greater than 10 GPa.

13. A composite material as claimed in claim 11 having a flexural modulus greater than 12 GPa.

14. A composite material as claimed in claim 11 having a flexural modulus greater than 15 GPa.

15. A composite material as claimed in any one of claims 1 to 14 having a flexural strength between 50 and 150 MPa.

16. A composite material as claimed in claim 15 having a flexural strength greater than 60 MPa.

17. A composite material as claimed in claim 15 having a flexural strength greater than 80 MPa.

18. A composite material as claimed in claim 15 having a flexural strength greater than 100 MPa.

19. A composite material as claimed in any one of claims 1 to 18 having a flexural ductility between 0.5 and 10%.

20. A composite material as claimed in claim 19 having a flexural ductility between 0.5 and 7%.

21. A composite material as claimed in claim 20 having a flexural ductility between 0.5 and 4%.

22. A composite material as claimed in any one of the preceding claims wherein the fibrous polymeric material is a polyolefin.

23. A composite material as claimed in claim 22 wherein the polyolefin is polyethylene.

24. A composite material as claimed in claim 22 wherein the polyethylene is of high modulus.

25. A composite material as claimed in any one of claims 1 to 24 characterised in that it includes a recrystallized melt phase of the polymeric material which has a melting point less than that of the oriented fibre and which binds the fibre material together.

26. A method for producing a composite material comprising combining oriented polymeric fibres with an inorganic filler material and compressing the combined material using hot compaction characterised in that it includes
(i) combining the polymeric material with the filler material and maintaining them at a contact pressure at which at least some of the fibres are in intimate contact with each other,
(ii) heating the combined material at an elevated temperature sufficient to melt only a proportion of the polymeric fibre and
(iii) compressing the heated combined material at a compaction pressure.

27. A method as claimed in claim 26 characterised in that the combining is carried out by mixing the materials.

28. A method as claimed in claim 26 wherein the contact pressure and compaction pressure are the same and this allows preferential surface melting of the fibres.

29. A method as claimed in claim 26 characterised in that the compaction pressure is higher than the contact pressure.

30. A method as claimed in claim 26 characterised in that the contact pressure is between 0.5 and 4 Mpa.

31. A method as claimed in any one of the preceding method claims characterised in that the proportion of the fibre that melts includes the surface and is from 5 to 95% by weight of the fibre.

32. A method as claimed in claim 31 characterised in that the proportion of the fibre is from 5 to 50% by weight of the fibre.

33. A method as claimed in claim 26 characterised in that the compressed mixture is cooled such that on cooling the melted part of the fibrous polymeric material forms a three dimensional matrix binding the fibrous material and filler material together.

34. A method as claimed in any one of claims 26 to 33 characterised in that-the mixture is maintained at a temperature at least that which an extrapolation of the leading edge of the endotherm of the fibrous material measured by differential scanning calorimetry intersectsthe temperature axis.

35. A method as claimed in claim 26 characterised in that the temperature at which the mixture is maintained is less than the peak temperature of melting of the polymer fibres as measured by differential scanning calorimetry.

36. A method as claimed in any one of claims 26 to 35 characterised in that the mixture is maintained at 0.5 to 4 MPa during (i) and (ii) prior to compressing at a compaction pressure.

37. A method as claimed in claim 36 characterised in that the mixture is maintained at between 0.5 and 2 MPa prior to compressing at a compaction pressure.

38. A method as claimed in any one of claims 26 to 37 characterised in that the fibres are in the form of continuous fibres that have been chopped into smaller lengths.

39. A method as claimed in any one of claims 26 to 38 characterised in that the temperature at which the mixture is maintained is between 1 and 10° C. below the melting point of the polymeric material.

40. A method as claimed in claim 39 characterised in that the temperature is between 1 and 5° C. below the melting point of the polymeric material.

41. A method as claimed in any one of claims 26 to 40 characterised in that the compacted material is subjected to extrusion.

42. A method as claimed in claim 41 characterised in that the extrusion step is carried out by hydrostatic extrusion.

43. A method as claimed in claim 41 or 42 characterised in that the product from step (iii) or the extrusion step is powderised then reprocessed as in steps (i) to (iii).

44. A method as claimed in claim 43 characterised in that the reprocessed material is then subjected to extrusion.

45. A method as claimed in claim 44 characterised in that the extrusion is hydrostatic extrusion.

46. A method as claimed in claim 42 or claim 45 wherein the hydrostatic extrusion step is performed by (iv) placing a billet of the material in contact with a die orifice while being surrounded by a fluid medium, (v) heating then fluid and the billet to a temperature below the melting point of the polymeric component of the material and (vi) applying pressure to the fluid such as to cause the billet to be extruded through the die.

47. A method as claimed in claim 46 characterised in that the die is a convergent die.

48. A method as claimed in claim 46 or 47 wherein the extrusion ratio of the extruded product is 3:1 or more.

49. A method as claimed in any one of claims 41 to 48 wherein the extrusion ratio is 7:1 or more.

50. A method as claimed in any one of claims 41 to 49 wherein the extrusion ratio is at least 11:1.

51. A method as claimed in claim 42 or 45 characterised in that the fluid is an oil.

52. A method as claimed in any one of claims 26 to 51 characterised in that the compaction pressure used in step (iii) is from 5 to 1000 MPa.

53. A method as claimed in claim 52 characterised in that the compaction pressure used in step (iii) is from 20 to 500 Mpa.

54. A method as claimed in claim 53 characterised in that the compaction pressure is from 40 to 80 MPa.

55. A composite or method as claimed in any one of claims 1 to 54 wherein the polymer is a homo or co-polymer of a polyolefin.

56. A composite or method as claimed in claim 55 wherein the polymer has a weight average molecular weight of 50,000 to 3,000,000.

57. A composite or method as claimed in claim 56 wherein the polymer has a weight average molecular weight of 100,000 to 3,000,000.

58. A composite or method as claimed in claim 57 wherein the polymer has a weight average molecular weight of 500,000 to 3,000,000.

59. A composite or method as claimed in any one of claims 55 to 59 characterised in that the fibre is gel or melt spun fibre.

60. A structural material comprising a composite as claimed in or provided by a method as claimed in any one of the preceding claims.

61. A prosthesis comprising a material as claimed in claim 61.
